SEQUENCE LISTING (23 sequences)

SEQ ID NO: 1 - DNA, 925 bp, unidentified
  source 1..925: mol_type = other DNA
atggtgagtggcagtaaagcgggcgtttcgcctcatcgcgaaatagaagtaatgagacaatccattgacgatcacctggctggcctgttacctgaaaccgacagccaggatatcgtcagccttgcgatgcgtgaaggcgtcatggcacccggtaaacggatccgtccgctgctgatgctgctggccgcccgcgacctccgctaccagggcagtatgcctacgctgctcgatctcgcctgcgccgttgaactgacccataccgcgtcgctgatgctcgacgacatgccctgcatggacaacgccgagctgcgccgcggtcagcccactacccacaaaaaatttggtgagagcgtggcgatccttgcctccgttgggctgctctctaaagcctttggtctgatcgccgccaccggcgatctgccgggggagaggcgtgcccaggcggtcaacgagctctctaccgccgtgggcgtgcagggcctggtactggggcagtttcgcgatcttaacgatgccgccctcgaccgtacccctgacgctatcctcagcaccaaccacctcaagaccggcattctgttcagcgcgatgctgcagatcgtcgccattgcttccgcctcgtcgccgagcacgcgagagacgctgcacgccttcgccctcgacttcggccaggcgtttcaactgctggacgatctgcgtgacgatcacccggaaaccggtaaagatcgcaataaggacgcgggaaaatcgacgctggtcaaccggctgggcgcagacgcggcccggcaaaagctgcgcgagcatattgattccgccgacaaacacctcacttttgcctgtccgcagggcggcgccatccgacagtttatgcatctgtggtttggccatcaccttgccgactggtcaccggtcatgaaaatcgccctga

SEQ ID NO: 2 - DNA, 1479 bp, unidentified
  source 1..1479: mol_type = other DNA
atgaaaaaaaccgttgtgattggcgcaggctttggtggcctggcgctggcgattcgcctgcaggcggcagggatcccaaccgtactgctggagcagcgggacaagcccggcggtcgggcctacgtctggcatgaccagggctttacctttgacgccgggccgacggtgatcaccgatcctaccgcgcttgaggcgctgttcaccctggccggcaggcgcatggaggattacgtcaggctgctgccggtaaaacccttctaccgactctgctgggagtccgggaagaccctcgactatgctaacgacagcgccgagcttgaggcgcagattacccagttcaacccccgcgacgtcgagggctaccggcgctttctggcttactcccaggcggtattccaggagggatatttgcgcctcggcagcgtgccgttcctctcttttcgcgacatgctgcgcgccgggccgcagctgcttaagctccaggcgtggcagagcgtctaccagtcggtttcgcgctttattgaggatgagcatctgcggcaggccttctcgttccactccctgctggtaggcggcaaccccttcaccacctcgtccatctacaccctgatccacgcccttgagcgggagtggggggtctggttccctgagggcggcaccggggcgctggtgaacggcatggtgaagctgtttaccgatctgggcggggagatcgaactcaacgcccgggtcgaagagctggtggtggccgataaccgcgtaagccaggtccggctggcggatggtcggatctttgacaccgacgccgtagcctcgaacgctgacgtggtgaacacctataaaaagctgctcggccaccatccggtggggcagaagcgggcggcagcgctggagcgcaagagcatgagcaactcgctgtttgtgctctacttcggcctgaaccagcctcattcccagctggcgcaccataccatctgttttggtccccgctaccgggagctgatcgacgagatctttaccggcagcgcgctggcggatgacttctcgctctacctgcactcgccctgcgtgaccgatccctcgctcgcgcctcccggctgcgccagcttctacgtgctggccccggtgccgcatcttggcaacgcgccgctggactgggcgcaggaggggccgaagctgcgcgaccgcatctttgactaccttgaagagcgctatatgcccggcctgcgtagccagctggtgacccagcggatctttaccccggcagacttccacgacacgctggatgcgcatctgggatcggccttctccatcgagccgctgctgacccaaagcgcctggttccgcccgcacaaccgcgacagcgacattgccaacctctacctggtgggcgcaggtactcaccctggggcgggcattcctggcgtagtggcctcggcgaaagccaccgccagcctgatgattgaggatctgcaatga

SEQ ID NO: 3 - DNA, 930 bp, unidentified
  source 1..930: mol_type = other DNA
atgagccaaccgccgctgcttgaccacgccacgcagaccatggccaacggctcgaaaagttttgccaccgctgcgaagctgttcgacccggccacccgccgtagcgtgctgatgctctacacctggtgccgccactgcgatgacgtcattgacgaccagacccacggcttcgccagcgaggccgcggcggaggaggaggccacccagcgcctggcccggctgcgcacgctgaccctggcggcgtttgaaggggccgagatgcaggatccggccttcgctgcctttcaggaggtggcgctgacccacggtattacgccccgcatggcgctcgatcacctcgacggctttgcgatggacgtggctcagacccgctatgtcacctttgaggatacgctgcgctactgctatcacgtggcgggcgtggtgggtctgatgatggccagggtgatgggcgtgcgggatgagcgggtgctggatcgcgcctgcgatctggggctggccttccagctgacgaatatcgcccgggatattattgacgatgcggctattgaccgctgctatctgcccgccgagtggctgcaggatgccgggctgaccccggagaactatgccgcgcgggagaatcgggccgcgctggcgcgggtggcggagcggcttattgatgccgcagagccgtactacatctcctcccaggccgggctacacgatctgccgccgcgctgcgcctgggcgatcgccaccgcccgcagcgtctaccgggagatcggtattaaggtaaaagcggcgggaggcagcgcctgggatcgccgccagcacaccagcaaaggtgaaaaaattgccatgctgatggcggcaccggggcaggttattcgggcgaagacgacgagggtgacgccgcgtccggccggtctttggcagcgtcccgtttag

SEQ ID NO: 4 - DNA, 5230 bp, unidentified
  source 1..5230: mol_type = other DNA
ggggtctgacgctcagtggaacgaaaactcacgttaagggattttgctctagaatgaaaggggattttatgcttcagaacgctcggttgccgccgggcgttttttatactagttcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgttttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatcaaaatcgtctccctccgtttgaatatttgattgatcgtaaccagatgaagcactctttccactatccctacagtgttatggcttgaacaatcacgaaacaataattggtacgtacgatctttcagccgactcaaacatcaaatcttacaaatgtagtctttgaaagtattacatatgtaagatttaaatgcaaccgttttttcggaaggaaatgatgacctcgtttccaccggaattagcttggtaccagctattgtaacataatcggtacgggggtgaaaaagctaacggaaaagggagcggaaaagaatgatgtaagcgtgaaaaattttttaaaaaatctcttgacattggaagggagatatgttattataagaattgcggaattgtgagcggataacaattcataattgtgagcggataacaattcaaccccaaaggaggtgggatccgtgatagcggtaccctcgaagctgtcaccggatgtgctttccggtctgatgagtccgtgaggacgaaacagcctctacaaataattttgtttaatagagggtaagtaaaagaaaggaggtttattttacaaaacatatttcaacacaatacaaatgggttagttaaaaaagcaggccttctaaaggtctgctttttttatttgattatgtaatttttaatgccaggatgccaataagccataacctcaaatgcaccatttgcaacctcgtcatcttcttcctcaatcttgaccagatcgccgtcctccgcatcaccaagattcagctctttatgtatctccttcaaaatgccaccgtatccaattaaccttcgagctgccaacgcatcatccaagtaaagcaccgtgttcagattgtcttcagtcaccttattaccgcgcacacaatccgtatccttaaccggatatttagagatttcgcgaacagctttttgctccatcattgcgttccgcacatcgttttcaatctgttcagcgtcaatcttagctttacctttcactcgacgaatatcgacaattggagtgtaatccaatttcatcgcctttttccaaaggctcgtccactccgcctgcttaatatagtttttcccaaaataatttttccttactggtatcaacacatgaaaatgaggatgatatgtatcttcttcatgatttttggtaatctctaaagctctgaaaaatccaagaaccgaagtttttacttttttgtactggaacagtttcctaaagccttccatcatcgcagaaatttgtggcttcagccgttctccctttacatttcgaatcgtcagcgtgagaaaaatccatccgcagccgtactgtctattggcttcctctacgatcaacttattgtgataagcaatttttaacgacctgcgccacgcacacatcggacataacctcactttacaaaaatgggcttgatacagttttaacttgcccgtctccgggtctctcttaaacgaaagatactctgcacaactaattagtttttcagcctttttgccatagtaaggtgccccaatcttactctctaacgcttcgtaatgctccgccatgaggttcgtccgtctctttttccccttccaatcccgctttttacctgttgcggttttatcttcgaggatgctataatcattttcagatgaataaatcaacaaaaaaactccttctgagctagttctctagcattctattattttgattcgacaccttaataatagcagaaggagtttttacctgtcaaagaaccatcaaacccttgatacacaaggctttgacctaattttgaaaaatgatgttgtttctatatagtatcaagataagaaagaaaaggatttttcgctacgctcaaatcctttaaaaaaacacaaaagaccacattttttaatgtggtctttattcttcaactaaagcacggcgcgcccctccttgacactgaatttagcatgtgatataattaacttaatattctacccaagcttataaaagagcactgttgggcgtgagtggaggcgccggaaaaaagcatcgaaaaaaggaggaaaaaaaatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatatgagataatgccgactgtactttttacagtcggttttctaatgtcactaacctgccccgttagtcgccattcgccagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctac

SEQ ID NO: 5 - DNA, 3582 bp, unidentified
  source 1..3582: mol_type = other DNA
atgacagatcaaaacagaaaagagcttttgcataaaaccggggaactttataagcaatttattgagaatcaagatgaacaaagggctgctaaactggctgcggtcatgaaaaaagcggctgatgaagaggtttatatcgcgtttaccgggcattattcagcggggaaatcgtcactgctgaactgcctgttaatggagaatattttgccgacaagcccgattccgacaagcgctaaccttgttgtgatcagaaacggggaaaagcgagttcggcttcatacaacagacggagcgtgtgctgagctggaagggacttaccagaaagacaaggtgcagcaatattgtaaagacggggaacagattgaaagcgtcgaaatttttgacaggtatactgagatcgattccggggttgcgtatattgatactccagggatagattcaacagatgatgcgcattttctatcggctgcttctattcttcaccaagcggatgcgctgttttacgtcgttcactataatcacgtgcacgctgaggaaaatgtaaagtttctcagatcaataaaagaaagcattccgaatgtgtattttatcgtcaatcaaattgaccgccatgatgagacggagacgaaatttggagactatcaagcgcaggtagaggagatgctttgcaatgaagggatttcaagggaagcgctctattttacatcagtaacagaaccggatcacccgttcaatcagatgggggccctgagagaagagttaagcagaatagaacagcaatcaaaaagcaatatgcaggctttaaccgaacaaaaagtccgtaatctgctaaaggaacatacggaaatgctgaagaaagacgaaacaggcgctccgagtttcgctgaacagcttaatatccatactggactggtccaatcgctgcgtgatcaattggatgaggccgaaaaacaaatgactgaggcagagaagaggatgcaggaagaaatcaatcgtattttaaaaaacgccaacctgacgccttttgaaatgcgtgaattggcggctgcatttttagaatcacaagagccttcctttaaaaccggctttttcttttctaaagcgaaaactgcacaggaaagagataaaaggcggaatgcttttttctctgatgtggcaaaacgaacagaggccgaagcggattggcatatgatagacacgcttcataaactcgccaaggtatttgatgtctataccgctgaaagtgaaaagctgattcaggcctatcgcacaccgcttgatataagcatcattgaacacgcggtcaagcatggtgcagccttttcctctgaatatgtattgcaatatacaaaagacttggcagagctcatccggaaagaggcgaaaagagaagccgcagacatcataaaggtgctctccgctatggtgaaagagcgtgtttccaaagatgtgcaaacaattaacgaccggctcgtccaagaatcagaaaaacttgtctttcttcaagaacaggcacgtttagagaataatgcccgagagaaaaccgatcgcctgtgggccatttgggaagaagagtccgcatgcccaatgcatatagacacagaatggtttaaatcgaagaaaacaagagtggcggctccagagcagaaacaaggccgaagccagctgacggcgcagccgatgccgaaatcagaaatcaaaatggaacaagaaatgccgcttcaagaccaaatcaaacgcttttacactttgtctgacattttaggcgaatgcagcatgttgttaaagcaaacttcagcgtttcgtgaaagggtgaaaaggcttgaggaacggaaatttacactcgctctttttggaggctttagctcggggaaatcatcatttgccaatgcgcttgtcggcgaaagagtgctgccatcctcaccaacgccaaccacagcgacaattaacaagatcacaaagccaatcaatggcaacttaaacaaaacagcgaatgtagtgtttaagacagaggatgacttaacggctgaaattttgcagctcaccggaataccaaaggagccggcaggccgttccttcacagagaagtgggaaaaggcagtcaaaaaaaacaggctgcaggaagaacacgtgaaattgatcagcaatttcctgcttgcatacgagaagtatcagcagtatatacaggaacagaagaaattaacgattccgctctctgaattaaagccgtatgtagcagaggaaacaacggcctgtgctgtgaaagaggtaacggtctactatacatgcccgctcactgaaaaagggattacgatcgttgatacacccggcgcgagcagcatgaataaacggcatacagaactggcattccaatatatcaaagatgctgatgcatttttttatatgacttattatcagcactctttttcaaaaggagaccgttcatttttgcgaaagcttggacttgtgaaagaatcgctcagcatggataaaatgttttttattattaatgctgcagaccttgcaaaagataaaacagagctcgaaacagtgaccgattatgtcagcgcagagcttgtaaaagaaggggtttatgagccgcagctctttaccgtatcaagtaaggaggaactggtgggaaaaccggagtcattttataatcagttctcgaaagtaagaaaacacttagaccggtttatcgaagtggatgtaaaaaaagcgtcagcagcgcagctcagttcggaggcagacaaactatgcgaaactgtttttcagcttcaccaatcccagcatcagtcccgagaagagaaggaagcgcagaaacagtgccttatgctgtcatttgagcggacagcggccgatatcgagaaacggcggaactcaaaaaccatcattgaaaaagtaaaaaaagatacaagagagcagctatatcacattgcacagcgattatcatattttgcaaatgatttattaaaatcggcatttcatcccgggctgcaaaacggggattggaaaaagaacgtcagtaaagcgatgacaacagccttacatgaatatctatttgaatatattcaggaaatcaaaacgcttgatgttcgcatgagcggtttcatcgaacggcatatcaatgaagaatggctggatcattttcaaaaaacattaaacgaggacggttatttttccgtttatgcgggtgatcagcattcgaacggcatacagttgaaggaagttgaaccggaaatagaagaacgtgcttttgagcaggaactaaaagagatcaaatcaccgaagcagttttttgagcagaaaggaaaagcgacctttatcgaagctgtacggatgaaacttacgaaaatcacagaggcatggataaaaaatgaagaagaaagcctgatatctcattacactgctcacctcagacgcttacaggaagatatgggtgaaaaagcaatcgcgcaaataacggatcaaaaagagacatatttaagaggatatgcggaaggggaacatgcgaaagagattgaaatggcgtatcaggcgtgcatctcctggaaaaattcagacaatacaataaaaatgtaa

SEQ ID NO: 6 - DNA, 8176 bp, unidentified
  source 1..8176: mol_type = other DNA
tttgtctgcaactgaaaagtttataccttacctggaacaaatggttgaaacatacgaggctaatatcggcttattaggaatagtccctgtactaataaaatcaggtggatcagttgatcagtatattttggacgaagctcggaaagaatttggagatgacttgcttaattccacaattaaattaagggaaagaataaagcgatttgatgttcaaggaatcacggaagaagatactcatgataaagaagctctaaaactattcaataaccttacaatggaattgatcgaaagggtggaaggttaatggtacgaaaattaggggatctacctagaaagccacaaggcgataggtcaagcttaaagaacccttacatggatcttacagattctgaaagtaaagaaacaacagaggttaaacaaacagaaccaaaaagaaaaaaagcattgttgaaaacaatgaaagttgatgtttcaatccataataagattaaatcgctgcacgaaattctggcagcatccgaagggaattcatattacttagaggatactattgagagagctattgataagatggttgagacattacctgagagccaaaaaactttttatgaatatgaattaaaaaaaagaaccaacaaaggctgagacagactccaaacgagtctgtttttttaaaaaaaatattaggagcattgaatatatattagagaattaagaaagacatgggaataaaaatattttaaatccagtaaaaatatgataagattatttcagaatatgaagaactctgtttgtttttgatgaaaaaacaaacaaaaaaaatccacctaacggaatctcaatttaactaacagcggccaaactgagaagttaaatttgagaaggggaaaaggcggatttatacttgtatttaactatctccattttaacattttattaaaccccatacaagtgaaaatcctcttttacactgttcctttaggtgatcgcggagggacattatgagtgaagtaaacctaaaaggaaatacagatgaattagtgtattatcgacagcaaaccactggaaataaaatcgccaggaagagaatcaaaaaagggaaagaagaagtttattatgttgctgaaacggaagagaagatatggacagaagagcaaataaaaaacttttctttagacaaatttggtacgcatataccttacatagaaggtcattatacaatcttaaataattacttctttgatttttggggctattttttaggtgctgaaggaattgcgctctatgctcacctaactcgttatgcatacggcagcaaagacttttgctttcctagtctacaaacaatcgctaaaaaaatggacaagactcctgttacagttagaggctacttgaaactgcttgaaaggtacggttttatttggaaggtaaacgtccgtaataaaaccaaggataacacagaggaatccccgatttttaagattagacgtaaggttcctttgctttcagaagaacttttaaatggaaaccctaatattgaaattccagatgacgaggaagcacatgtaaagaaggctttaaaaaaggaaaaagagggtcttccaaaggttttgaaaaaagagcacgatgaatttgttaaaaaaatgatggatgagtcagaaacaattaatattccagaggccttacaatatgacacaatgtatgaagatatactcagtaaaggagaaattcgaaaagaaatcaaaaaacaaatacctaatcctacaacatcttttgagagtatatcaatgacaactgaagaggaaaaagtcgacagtactttaaaaagcgaaatgcaaaatcgtgtctctaagccttcttttgatacctggtttaaaaacactaagatcaaaattgaaaataaaaattgtttattacttgtaccgagtgaatttgcatttgaatggattaagaaaagatatttagaaacaattaaaacagtccttgaagaagctggatatgttttcgaaaaaatcgaactaagaaaagtgcaataaactgctgaagtatttcagcagttttttttatttagaaatagtgaaaaaaatataatcagggaggtatcaatatttaatgagtactgatttaaatttatttagactggaattaataattaacacgtagactaattaaaatttaatgagggataaagaggatacaaaaatattaatttcaatccctattaaattttaacaagggggggattaaaatttaattagaggtttatccacaagaaaagaccctaataaaatttttactagggttataacactgattaatttcttaatgggggagggattaaaatttaatgacaaagaaaacaatcttttaagaaaagcttttaaaagataataataaaaagagctttgcgattaagcaaaactctttactttttcattgacattatcaaattcatcgatttcaaattgttgttgtatcataaagttaattctgttttgcacaaccttttcaggaatataaaacacatctgaggcttgttttataaactcagggtcgctaaagtcaatgtaacgtagcatatgatatggtatagcttccacccaagttagcctttctgcttcttctgaatgtttttcatatacttccatgggtatctctaaatgattttcctcatgtagcaaggtatgagcaaaaagtttatggaattgatagttcctctctttttcttcaacttttttatctaaaacaaacactttaacatctgagtcaatgtaagcataagatgtttttccagtcataatttcaatcccaaatcttttagacagaaattctggacgtaaatcttttggtgaaagaatttttttatgtagcaatatatccgatacagcaccttctaaaagcgttggtgaatagggcattttacctatctcctctcattttgtggaataaaaatagtcatattcgtccatctacctatcctattatcgaacagttgaactttttaatcaaggatcagtcctttttttcattattcttaaactgtgctcttaactttaacaactcgatttgtttttccagatctcgagggtaactagcctcgccgatcccgcaagaggcccggcagtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcatgcttaagttattggtatgactggttttaagcgcaaaaaaagttgctttttcgtacctattaatgtatcgttttagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaaagaccacattaaaaaatgtggtcttttgtgtttttttaaaggatttgagcgtagcgaaaaatccttttctttcttatcttgataataagggtaactattgccgatcgtccattccgacagcatcgccagtcactatggcgtgctgctagcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcaggccttaactcacattaattgcgttgcgctctagaatgaaaggggattttatgcttcagaacgctcggttgccgccgggcgttttttatactagttcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgttttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatcaaaatcgtctccctccgtttgaatatttgattgatcgtaaccagatgaagcactctttccactatccctacagtgttatggcttgaacaatcacgaaacaataattggtacgtacgatctttcagccgactcaaacatcaaatcttacaaatgtagtctttgaaagtattacatatgtaagatttaaatgcaaccgttttttcggaaggaaatgatgacctcgtttccaccggaattagcttggtaccagctattgtaacataatcggtacgggggtgaaaaagctaacggaaaagggagcggaaaagaatgatgtaagcgtgaaaaattttttaaaaaatctcttgacattggaagggagatatgttattataagaattgcggaattgtgagcggataacaattcataattgtgagcggataacaattcaaccccaaaggaggtgggatccgtgatagcggtaccctcgaagctgtcaccggatgtgctttccggtctgatgagtccgtgaggacgaaacagcctctacaaataattttgtttaatagagggtaagtaaaagaaaggaggtttatttttaactgcaggtcgacgtccccggggcagcccgcctaatgagcgggcttttttcacgtcacgcgtccatggagatc

SEQ ID NO: 7 - protein, 1193 aa, unidentified
MTDQNRKELLHKTGELYKQFIENQDEQRAAKLAAVMKKAADEEVYIAFTGHYSAGKSSLLNCLLMENILPTSPIPTSANLVVIRNGEKRVRLHTTDGACAELEGTYQKDKVQQYCKDGEQIESVEIFDRYTEIDSGVAYIDTPGIDSTDDAHFLSAASILHQADALFYVVHYNHVHAEENVKFLRSIKESIPNVYFIVNQIDRHDETETKFGDYQAQVEEMLCNEGISREALYFTSVTEPDHPFNQMGALREELSRIEQQSKSNMQALTEQKVRNLLKEHTEMLKKDETGAPSFAEQLNIHTGLVQSLRDQLDEAEKQMTEAEKRMQEEINRILKNANLTPFEMRELAAAFLESQEPSFKTGFFFSKAKTAQERDKRRNAFFSDVAKRTEAEADWHMIDTLHKLAKVFDVYTAESEKLIQAYRTPLDISIIEHAVKHGAAFSSEYVLQYTKDLAELIRKEAKREAADIIKVLSAMVKERVSKDVQTINDRLVQESEKLVFLQEQARLENNAREKTDRLWAIWEEESACPMHIDTEWFKSKKTRVAAPEQKQGRSQLTAQPMPKSEIKMEQEMPLQDQIKRFYTLSDILGECSMLLKQTSAFRERVKRLEERKFTLALFGGFSSGKSSFANALVGERVLPSSPTPTTATINKITKPINGNLNKTANVVFKTEDDLTAEILQLTGIPKEPAGRSFTEKWEKAVKKNRLQEEHVKLISNFLLAYEKYQQYIQEQKKLTIPLSELKPYVAEETTACAVKEVTVYYTCPLTEKGITIVDTPGASSMNKRHTELAFQYIKDADAFFYMTYYQHSFSKGDRSFLRKLGLVKESLSMDKMFFIINAADLAKDKTELETVTDYVSAELVKEGVYEPQLFTVSSKEELVGKPESFYNQFSKVRKHLDRFIEVDVKKASAAQLSSEADKLCETVFQLHQSQHQSREEKEAQKQCLMLSFERTAADIEKRRNSKTIIEKVKKDTREQLYHIAQRLSYFANDLLKSAFHPGLQNGDWKKNVSKAMTTALHEYLFEYIQEIKTLDVRMSGFIERHINEEWLDHFQKTLNEDGYFSVYAGDQHSNGIQLKEVEPEIEERAFEQELKEIKSPKQFFEQKGKATFIEAVRMKLTKITEAWIKNEEESLISHYTAHLRRLQEDMGEKAIAQITDQKETYLRGYAEGEHAKEIEMAYQACISWKNSDNTIKM

SEQ ID NO: 8 - DNA, 40 bp, synthetic construct
  source 1..40: mol_type = other DNA
aaaataaacctcctttcttttacttaccctctattaaaca

SEQ ID NO: 9 - DNA, 32 bp, synthetic construct
  source 1..32: mol_type = other DNA
acaaaacatatttcaacacaatacaaatgggt

SEQ ID NO: 10 - DNA, 42 bp, synthetic construct
  source 1..42: mol_type = other DNA
aaagaaaggaggtttattttatggtgagtggcagtaaagcgg

SEQ ID NO: 11 - DNA, 48 bp, synthetic construct
  source 1..48: mol_type = other DNA
attatcctccttaccctctatcagggcgattttcatgaccggtgacca

SEQ ID NO: 12 - DNA, 48 bp, synthetic construct
  source 1..48: mol_type = other DNA
tagagggtaaggaggataattttatgaaaaaaaccgttgtgattggcg

SEQ ID NO: 13 - DNA, 43 bp, synthetic construct
  source 1..43: mol_type = other DNA
ataatcctcctttccctctatcattgcagatcctcaatcatca

SEQ ID NO: 14 - DNA, 43 bp, synthetic construct
  source 1..43: mol_type = other DNA
tagagggaaaggaggattatattatgagccaaccgccgctgct

SEQ ID NO: 15 - DNA, 40 bp, synthetic construct
  source 1..40: mol_type = other DNA
tgtgttgaaatatgttttgtctaaacgggacgctgccaaa

SEQ ID NO: 16 - DNA, 26 bp, synthetic construct
  source 1..26: mol_type = other DNA
cagtggaacgaaaactcacgttaagg

SEQ ID NO: 17 - DNA, 42 bp, synthetic construct
  source 1..42: mol_type = other DNA
tcctggcattaaaaattacataatcaaataaaaaaagcagac

SEQ ID NO: 18 - DNA, 20 bp, synthetic construct
  source 1..20: mol_type = other DNA
taactgcaggtcgacgtccc

SEQ ID NO: 19 - DNA, 41 bp, synthetic construct
  source 1..41: mol_type = other DNA
aaaataaacctcctttcttttacttaccctctattaaacaa

SEQ ID NO: 20 - DNA, 51 bp, synthetic construct
  source 1..51: mol_type = other DNA
aaagaaaggaggtttattttatgacagatcaaaacagaaaagagcttttgc

SEQ ID NO: 21 - DNA, 60 bp, synthetic construct
  source 1..60: mol_type = other DNA
gggacgtcgacctgcagttattacatttttattgtattgtctgaatttttccaggagatg

SEQ ID NO: 22 - DNA, 32 bp, synthetic construct
  source 1..32: mol_type = other DNA
ccacctgattttattagtacagggactattcc

SEQ ID NO: 23 - DNA, 23 bp, synthetic construct
  source 1..23: mol_type = other DNA
ggtacgtacgatctttcagccga

(19) United States
(12) Patent Application Publication
(10) Pub. No.: US 2026/0347754 A1
(43) Pub. Date: Nov. 5, 2026

(54) BSUBTILIS SUBTILIS STRAIN FOR PRODUCTION OF LYCOPENE BY EXTRACELLULAR ORGANELLE

(71) Applicants: JITRI FUTURE FOOD TECHNOLOGY INSTITUTE CO., LTD., Wuxi (CN); JIANGNAN UNIVERSITY, Wuxi (CN)

(72) Inventors: Xueqin LV, Wuxi (CN); Long LIU, Wuxi (CN); Jian CHEN, Wuxi (CN); Guocheng DU, Wuxi (CN); Suwei LI, Wuxi (CN); Ziyang HUANG, Wuxi (CN)

(21) Appl. No.: 19/362,827

(22) Filed: Oct. 20, 2025

Related U.S. Application Data
(63) Continuation of application No. PCT/CN2025/101985, filed on Jun. 19, 2025.

(30) Foreign Application Priority Data
Apr. 25, 2025 (CN) 202410505890.5

(51) Int. Cl. C12N 1/20; C12N 9/02; C12N 9/10; C12N 15/75; C12P 5/00; C12R 1/125

(52) U.S. Cl. CPC C12N 1/20; C12N 9/001; C12N 9/1085; C12N 15/75; C12P 5/007; C12N 2800/101; C12R 2001/125; C12Y 205/01029; C12Y 205/01032

(57) ABSTRACT

The present disclosure provides a Bacillus subtilis strain for production of lycopene by an extracellular organelle. Specifically, in the present disclosure, synthesized crtE, crtI and crtB genes are integrated into plasmid PADK using gene recombination technology to construct plasmid PADK-FQHS, and the plasmid is transformed into Bacillus subtilis to construct strain BS PADK-FQHS. Subsequently, a dyna gene derived from Bacillus subtilis is integrated into plasmid PHT01 using gene recombination technology to construct plasmid PHT-dyna, and the plasmid is transformed into the strain BS PADK-FQHS to construct strain BS PADK-FQHS PHT-dyna. The lycopene yields in strains BS PADK-FQHS and BS PADK-FQHS PHT-dyna are detected, which demonstrates the promoting effect of nanotubes on the production of lycopene by Bacillus subtilis, with the yield increased by 67.1%.

This application is a Continuation of PCT/CN2025/101985, filed on Jun. 19, 2025, which claims priority to Chinese Patent Application No. 202410505890.5, filed on Apr. 25, 2025, which is incorporated by reference for all purposes as if fully set forth herein.

A Sequence Listing XML file named “10015_0184.xml” created on Oct. 20, 2025 and having a size of 42,762 bytes, is filed concurrently with the specification. The sequence listing contained in the XML file is part of the specification and is herein incorporated by reference in its entirety.

### FIELD OF THE INVENTION

The present disclosure relates to a Bacillus subtilis strain for production of lycopene by an extracellular organelle, which belongs to the field of biotechnology.

### DESCRIPTION OF THE RELATED ART

Microbial metabolic engineering aims at the construction of stable and efficient cell factories in order to increase the yield of a product of interest or to endow cells with the ability to produce a new product. As a model microorganism, Bacillus subtilis has profound and extensive applications in biological manufacturing industries such as recombinant protein production and bioactive substance synthesis. Functional membrane microdomains (FMMs) are a class of microdomains with compact structures on the cell membrane of Bacillus subtilis, which are involved in life activities such as cell signaling, energy metabolism, and protein secretion. The FMMs divide the cell membrane into regions, so that the cellular processes are compartmentalized, and a platform is provided for the aggregation of proteins and enzymes. Since the membrane surface area of cells is relatively fixed, the proportion of FMMs on the cell membrane, after reaching a certain degree (nearly 50%), cannot be further increased by reconstitution of FMMs components. Therefore, effectively expanding the cell membrane surface area to thereby increase FMMs scaffolds is of great significance for constructing efficient Bacillus subtilis cell factories.

Lycopene is an isoprenoid compound, which is widely present in vegetables and fruits such as tomatoes and red carrots. As an antioxidant, lycopene can effectively improve the antioxidant capacity of its own tissues and cells. Lycopene, as a hydrophobic isoprenoid compound, is mainly stored in limited spaces such as cell membranes in cells, which will hinder its accumulation in large quantities. We hypothesized that this problem might be effectively addressed by increasing the membrane surface area.

In 2020, Jif̌i Pospíšil et al. cultivated Bacillus subtilis on poly-L-lysine-coated coverslips and glass slides and successfully induced the formation of nanotubes in Bacillus subtilis by applying a pressure of 80 kPa. Promoting the production of nanotubes in Bacillus subtilis can significantly increase the cell membrane surface area, thereby holding the promise of achieving efficient synthesis of a product of interest. However, in the work of Jiři Pospíšil et al., Bacillus subtilis can produce nanotubes only when cultivated on poly-L-lysine-coated coverslips and glass slides and subjected to a pressure of 80 kPa. When neither poly-L-lysine nor pressure is present, Bacillus subtilis cannot produce nanotubes. Given the current technical conditions in the biomanufacturing industry, this approach is not suitable for lycopene production. Therefore, the present disclosure aims at exploring how to increase the lycopene yield by expanding the cell membrane surface area.

### SUMMARY OF THE INVENTION

In order to solve the above problems, in the present disclosure, a Bacillus subtilis strain capable of efficiently expressing and synthesizing lycopene is constructed on the basis of molecular biological means, and on this basis, an extracellular organelle (nanotube) is applied to the strain, thereby greatly increasing the lycopene yield in the recombinant Bacillus subtilis strain.

A first object of the present disclosure is to provide a recombinant Bacillus subtilis strain for production of lycopene by an extracellular organelle, wherein the recombinant Bacillus subtilis strain heterologously expresses a coding gene dyna of a protein shown in SEQ ID NO. 7 and at least overexpresses a lycopene synthesis gene, and the lycopene synthesis gene includes a geranylgeranyl diphosphate synthase crtE, a phytoene desaturase gene crtI, and a phytoene synthase gene crtB.

Further, a sequence of the gene dyna is shown in SEQ ID NO. 5.

Further, a sequence of the geranylgeranyl diphosphate synthase gene crtE is preferably a sequence shown in SEQ ID NO. 1, a sequence of the phytoene desaturase gene crtI is preferably a sequence shown in SEQ ID NO. 2, and a sequence of the phytoene synthase gene crtB is preferably a sequence shown in SEQ ID NO. 3. Certainly, it will be understood by those skilled in the art that the recombinant strain protected by the present disclosure may further comprise other modifications, such as increasing the expression level of genes that can further increase the lycopene yield. The genes include, but are not limited to gene ERG10 encoding acetoacetyl-CoA thiolase, gene ERG13 encoding HMG-CoA synthase, gene tHMG1 encoding HMG-CoA reductase, gene ERG12 encoding mevalonate kinase, gene ERG8 encoding mevalonate-5-phosphate kinase, gene MVD1 encoding mevalonate pyrophosphate decarboxylase, gene IDI1 encoding isoprene pyrophosphate isomerase, etc.

Further, the heterologous expression and overexpression may be integrated expression or episomal expression.

Further, the episomal expression is preferably performed through plasmids.

Specifically, a vector for expressing the gene dyna includes, but is not limited to, a PHT01 plasmid, and a vector for expressing the lycopene synthesis gene includes, but is not limited to, a PADK plasmid.

Further, a host cell of the recombinant Bacillus subtilis strain may be any strain of Bacillus subtilis, such as Bacillus subtilis WB600 and Bacillus subtilis 168.

A second object of the present disclosure is to provide a method for constructing the recombinant Bacillus subtilis strain, comprising the following steps:
S1, constructing a recombinant plasmid or an integrated fragment comprising gene dyna, and constructing recombinant plasmids or integrated fragments, comprising gene crtE, gene crtI, and gene crtB, respectively or simultaneously; and S2, introducing the recombinant plasmids or integrated fragments obtained in S1 into a Bacillus subtilis host strain to obtain the recombinant Bacillus subtilis strain.

A third object of the present disclosure is to provide use of the recombinant Bacillus subtilis strain in the preparation of lycopene.

A fourth object of the present disclosure is to provide a method for producing lycopene, which comprises the following step: performing fermentation production using the recombinant Bacillus subtilis strain.

Further, a magnesium salt is added to a fermentation system (a magnesium salt is added to a seed medium and a fermentation medium).

Further, in the seed medium, a final concentration of magnesium ions is 3-7 mM, and in the fermentation medium, a final concentration of magnesium ions is 3-7 mM.

Further, the recombinant strain is inoculated into a seed medium to prepare a seed solution, and the seed solution is inoculated into a fermentation medium for fermentation production at 30-37 °C.

Further, the fermentation medium without a magnesium salt comprises: 10-15 g/L yeast powder, 4-8 g/L tryptone, 4-8 g/L (NH₄)₂SO₄, 10-15 g/L K₂HPO₄·3H₂O, 1-5 g/L KH₂PO₄, 1-5 g/L MgSO₄, and 40-80 g/L glucose.

The beneficial effects of the present disclosure are as follows:
(1) In order to obtain a genetically recombinant Bacillus subtilis strain capable of efficiently synthesizing lycopene, in the present disclosure, genes essential for Bacillus subtilis to synthesize lycopene are firstly integrated into a Bacillus subtilis host strain to construct strain BS PADK-FQHS, so as to endow Bacillus subtilis with the ability to synthesize lycopene. Then, on the basis of the strain, genes essential for Bacillus subtilis to synthesize an extracellular organelle (nanotube) are integrated to construct strain BS PADK-FQHS PHT-dyna, so as to endow Bacillus subtilis with the ability to produce a nanotube.
(2) In the present disclosure, the Bacillus subtilis strain capable of producing lycopene is successfully constructed, with the lycopene yield reaching 48.5 mg/L, and then the extracellular organelle (nanotube) is successfully applied to the production of lycopene. Compared with Bacillus subtilis not expressing the extracellular organelle (nanotube), the lycopene yield in the Bacillus subtilis expressing the extracellular organelle (nanotube) reaches 81.8 mg/L, showing an increase of 67.1%.

### BRIEF DESCRIPTION OF THE DRAWINGS

FIG. 1 shows the results of colony PCR of strain BS PADK-FQHS.

FIG. 2 shows the results of colony PCR of strain BS PADK-FQHS PHT-dyna.

FIG. 3 shows the results of the lycopene yield in strain BS PADK-FQHS and strain BS PADK-FQHS PHT-dyna.

FIG. 4 shows the production of bacterial nanotubes by strain BS PADK-FQHS PHT-dyna, in which the left panel is a bright field of view, and the right panel is a fluorescent field of view.

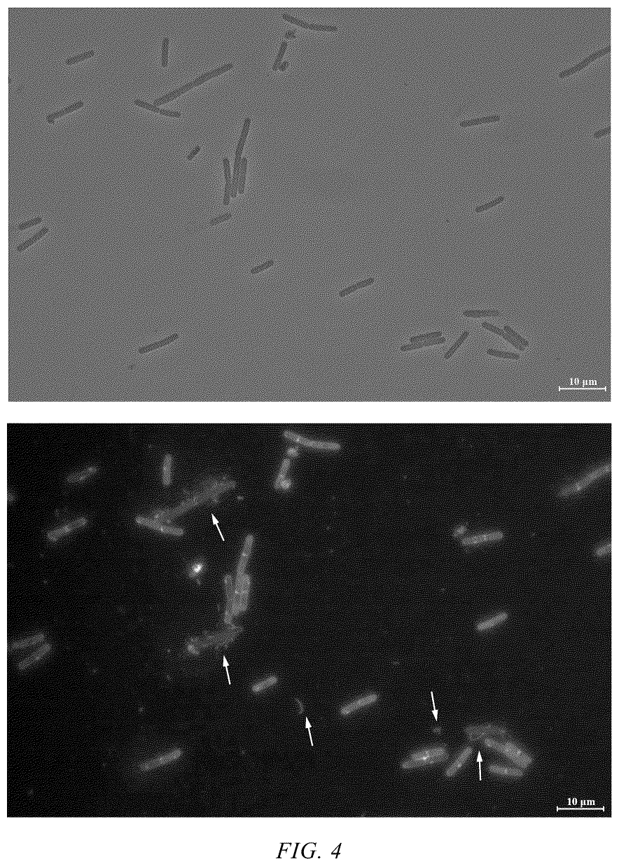

### DETAILED DESCRIPTION OF THE PREFERRED EMBODIMENTS

The present disclosure will be further described below with reference to the drawings and specific examples, such that those skilled in the art can better understand and implement the present disclosure. However, the examples given should not be construed as limiting the present disclosure.

The sequences of the primers involved in the present disclosure are as follows:

TABLE 1
Primer sequence

| Primer | |
|---|---|
| name | Sequence |
| PADK-T | AAAATAAACCTCCTTTCTTTTACTTACCCTCTATTAA |
| | ACA (SEQ ID NO. 8) |
| PADK-B | ACAAAACATATTTCAACACAATACAAATGGGT |
| | (SEQ ID NO. 9) |
| crtE-T | AAAGAAAGGAGGTTTATTTTATGGTGAGTGGCAGTAA |
| | AGCGG (SEQ ID NO. 10) |
| crtE-B | ATTATCCTCCTTACCCTCTATCAGGGCGATTTTCATG |
| | ACCGGTGACCA (SEQ ID NO. 11) |
| crtI-T | TAGAGGGTAAGGAGGATAATTTTATGAAAAAAACCGT |
| | TGTGATTGGCG (SEQ ID NO. 12) |
| crtI-B | ATAATCCTCCTTTCCCTCTATCATTGCAGATCCTCAA |
| | TCATCA (SEQ ID NO. 13) |
| crtB-T | TAGAGGGAAAGGAGGATTATATTATGAGCCAACCGCC |
| | GCTGCT (SEQ ID NO. 14) |
| crtB-B | TGTGTTGAAATATGTTTTGTCTAAACGGGACGCTGCC |
| | AAA(SEQ ID NO. 15) |
| PADK-JP- | CAGTGGAACGAAAACTCACGTTAAGG |
| 0 | (SEQ ID NO. 16) |
| PADK-JP- | TCCTGGCATTAAAAATTACATAATCAAATAAAAAAAG |
| 1 | CAGAC (SEQ ID NO. 17) |
| PHT01-M- | TAACTGCAGGTCGACGTCCC (SEQ ID NO. 18) |
| 0 | |
| PHT01-M- | AAAATAAACCTCCTTTCTTTTACTTACCCTCTATTAA |
| 1 | ACAA (SEQ ID NO. 19) |
| dyna-0 | AAAGAAAGGAGGTTTATTTTATGACAGATCAAAACAG |
| | AAAAGAGCTTTTGC (SEQ ID NO. 20) |
| dyna-1 | GGGACGTCGACCTGCAGTTATTACATTTTTATTGTAT |
| | TGTCTGAATTTTTCCAGGAGATG |
| | (SEQ ID NO. 21) |
| PHT-JP-0 | CCACCTGATTTTATTAGTACAGGGACTATTCC |
| | (SEQ ID NO. 22) |
| PHT-JP-1 | GGTACGTACGATCTTTCAGCCGA |
| | (SEQ ID NO. 23) |

### Example 1. Construction of Strain BS PADK-FQHS

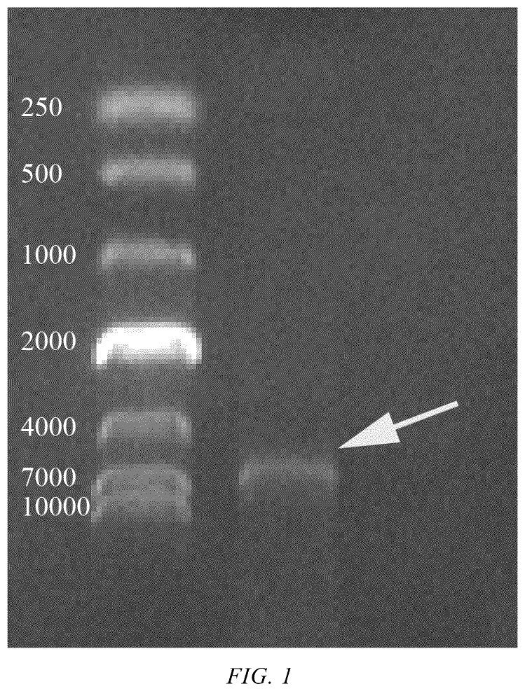

(1) a crte gene fragment (SEQ ID NO. 1) was obtained by amplification with a synthesized gene crte as a template and crte-T and crte-B as primers; a crtI gene fragment (SEQ ID NO. 2) was obtained by amplification with a synthesized gene crtI as a template and crtI-T and crtI-B as primers; a crtb gene fragment (SEQ ID NO. 3) was obtained by amplification with a synthesized gene crtb as a template and crtb-T and crtb-B as primers; a PADK plasmid fragment (SEQ ID NO. 4) was obtained by amplification with a plasmid PADK as a template and PADK-T and PADK-B as primers. Subsequently, the crte, crtI and crtb gene fragments were fused with a linearized vector PADK using Gibson Assembly® Master Mix from NEB (see product instructions for specific steps) to obtain a recombinant plasmid PADK-FQHS. The recombinant plasmid was sequenced, and the plasmid with correct sequencing results was stored at −20 °C.
(2) The PADK-FQHS plasmid constructed in step (1) was transformed into Bacillus subtilis 168 by electroporation under the conditions of a voltage of 2.5 kV and an electroporation time of 5 ms. After recovery at 37 °C for 5 h, the bacterial culture was spread on an LB plate containing kanamycin at a final concentration of 50 μg/mL for resistance selection, followed by incubation at 37 °C for 48 h. A single colony on the plate was picked, and colony PCR was performed with PADK-JP-0 and PADK-JP-1 as primers to obtain a verification fragment of 5243 bp (as shown in FIG. 1), which demonstrated that the plasmid was successfully transferred into Bacillus subtilis 168. This demonstrates that strain BS PADK-FQHS is successfully constructed.

### Example 2. Construction of Strain BS PADK-FQHS PHT-Dyna

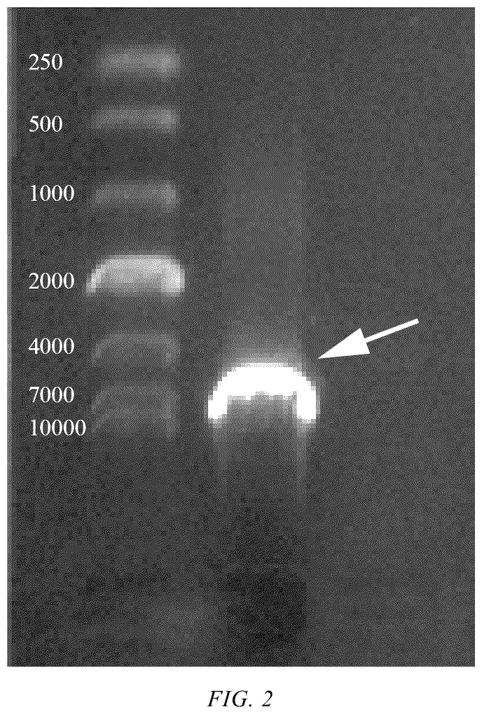

(1) A dyna gene fragment (SEQ ID NO. 5) was obtained by amplification with Bacillus subtilis 168 as a template and dyna-0 and dyna-1 as primers; a PHT01-GJ linearized vector (SEQ ID NO. 6) was obtained by amplification with a PHT01-M plasmid (MiaoLing) as a template and PHT01-M-0 and PHT01-M-1 as primers. Subsequently, the dyna gene fragment was fused with the linearized vector PHT01-GJ using Gibson Assembly® Master Mix from NEB (see product instructions for specific steps) to obtain a recombinant plasmid PHT01-dyna. The recombinant plasmid was sequenced, and the plasmid with correct sequencing results was stored at −20 °C.
(2) The PHT01-dyna plasmid constructed in step (1) was transformed into BS PADK-FQHS by electroporation under the conditions of a voltage of 2.5 kV and an electroporation time of 5 ms. After recovery at 37 °C for 5 h, the bacterial culture was spread on an LB plate containing chloramphenicol at a final concentration of 5 μg/mL and kanamycin at a final concentration of 50 μg/mL for resistance selection, followed by incubation at 37 °C for 48 h. A single colony on the plate was picked, and colony PCR was performed with PHT-JP-0 and PHT-JP-1 as primers to obtain a verification fragment of 4238 bp (as shown in FIG. 2), which demonstrated that the plasmid was successfully transferred into BS PADK-FQHS. This demonstrates that strain BS PADK-FQHS PHT-dyna is successfully constructed.

### Example 3. Verification of Lycopene Yields in Strain BS PADK-FQHS and Strain BS PADK-FQHS PHT-Dyna

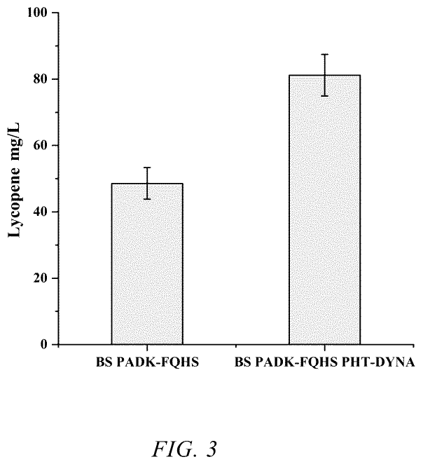

(1) Single colonies of the constructed strains BS PADK-FQHS and BS PADK-FQHS PHT-dyna were picked and inoculated into a liquid LB medium (10 μg/L peptone, 5 μg/L yeast powder, and 10 μg/L NaCl, additionally supplemented with 5 mM MgSO₄ and 1 mM IPTG) supplemented with 5 mM MgSO₄ and 1 mM IPTG at final concentrations, followed by incubation for 12 h. 1.5 mL of the prepared seed solution was inoculated into a 250 mL baffled shake flask containing 30 mL of lycopene medium (12 μg/L yeast powder, 6 μg/L tryptone, 6 μg/L (NH₄)₂SO₄, 12.5 μg/L K₂HPO₄·3H₂O, 2.5 μg/L KH₂PO₄, 3 μg/L MgSO₄, 60 μg/L glucose, 5 mM MgSO₄, and 1 mM IPTG), followed by fermentation for 72 h.
(2) 0.6 mL of the fermentation broth was pipetted into 0.6 mL of ethyl acetate. Cell disruption glass beads (0.1 mm in diameter) were added in a total volume of 0.1 mL. A cell disruptor was used at a linear speed of 4 m/s. Each disruption cycle lasted 60 s, and a total of 10 cycles of breaking were performed. Centrifugation was performed at 10,000 rpm for 10 min. The upper organic phase was pipetted, filtered through an organic filter membrane, and detected by liquid chromatography (C18 column, mobile phase: V_{acetonitrile}:Vₘₑₜₕₐₙₒₗ:Vᵢₛₒₚᵣₒₚₐₙₒₗ=5:3:2, flow rate: 1 mL/min, UV absorption wavelength: 450 nm). The results are shown in FIG. 3. As can be seen from the figure, the lycopene production of strain BS PADK-FQHS PHT-dyna is 167.1% of that of strain BS PADK-FQHS.

It is apparent that the above examples are merely illustrative for clear illustration and are not intended to limit the embodiments. Various changes and modifications can be made by those of ordinary skill in the art on the basis of the above description. It is unnecessary and impossible to enumerate all the embodiments herein. Obvious changes or modifications derived therefrom still fall within the scope of protection of the present disclosure.

What is claimed is:

1. A recombinant Bacillus subtilis strain for production of lycopene by an extracellular organelle, wherein the recombinant Bacillus subtilis strain heterologously expresses a coding gene dyna of a protein shown in SEQ ID NO. 7 and at least overexpresses a lycopene synthesis gene, and the lycopene synthesis gene comprises a geranylgeranyl diphosphate synthase gene crtE, a phytoene desaturase gene crtI, and a phytoene synthase gene crtB.

2. The recombinant Bacillus subtilis strain according to claim 1, wherein a sequence of the gene dyna is shown in SEQ ID NO. 5, a sequence of the gene crtE is shown in SEQ ID NO. 1, a sequence of the gene crtI is shown in SEQ ID NO. 2, and a sequence of the gene crtB is shown in SEQ ID NO. 3.

3. The recombinant Bacillus subtilis strain according to claim 1, wherein the heterologous expression and overexpression are integrated expression or episomal expression.

4. The recombinant Bacillus subtilis strain according to claim 3, wherein the episomal expression is performed through a plasmid, wherein a vector expressing the gene dyna comprises a PHT01 plasmid, and a vector expressing the lycopene synthesis gene comprises a PADK plasmid.

5. A method for constructing the recombinant Bacillus subtilis strain according to claim 1, comprising steps of:
S1, constructing a recombinant plasmid or an integrated fragment comprising gene dyna, and constructing a recombinant plasmid or an integrated fragment which comprises gene crtE, gene crtI, and gene crtB, respectively or simultaneously; and
S2, introducing the recombinant plasmid or integrated fragment obtained in S1 into a Bacillus subtilis host strain to obtain the recombinant Bacillus subtilis strain.

6. Use of the recombinant Bacillus subtilis strain according to claim 1 in the preparation of lycopene.

7. A method for producing lycopene, comprising a step of: performing fermentation using the recombinant Bacillus subtilis strain according to claim 1.

8. The method according to claim 7, wherein the recombinant strain is inoculated into a seed medium to prepare a seed solution, and the seed solution is inoculated into a fermentation medium for fermentation at 30-37 °C.

9. The method according to claim 7, wherein a magnesium salt is added to a fermentation system.

10. The method according to claim 9, wherein in the seed medium, a final concentration of magnesium ions is 3-7 mM; in the fermentation medium, a final concentration of magnesium ions is 3-7 mM.
